(19) United States
(12) Patent Application Publication
(10) Pub. No.: US 2021/0275046 A1
(43) Pub. Date: Sep. 9, 2021

(54) Peak Detection System and Method for Calculation of Signal-Derived Metrics

(71) Applicant: The Government of the United States as represented by the Secretary of the Army, Ft. Detrick, MD (US)

(72) Inventors: Nehemiah T. Liu, San Antonio, TX (US); Jose Salinas, San Antonio, TX (US)

(73) Assignee: The Government of the United States as represented by the Secretary of the Army, Ft. Detrick, MD (US)

(21) Appl. No.: 17/319,915

(22) Filed: May 13, 2021

Related U.S. Application Data
(62) Division of application No. 14/827,035, filed on Aug. 14, 2015.
(60) Provisional application No. 62/037,502, filed on Aug. 14, 2014.

(51) Int. Cl. A61B 5/024; A61B 5/00; A61B 5/0245; A61B 5/352

(52) U.S. Cl. CPC A61B 5/02405; A61B 5/7221; A61B 5/021; A61B 5/0245; A61B 5/352; A61B 5/7203

(57) ABSTRACT

The invention in at least one embodiment includes a method for providing a beat determination based on signals from a plurality of medical sensors in substantially real time, said method including: receiving in at least two analysis modules at least one signal (including an ECG signal and/or a pulse signal) from at least one medical sensor such that at least one analysis module receives one ECG signal and at least one analysis module receives one pulse signal; in each analysis module processing one signal through a plurality of detection algorithm modules to produce at least one quantitative decision, fusing the quantitative decisions together with a fusion module, and outputting at least one detected peak from the fusion module to an aggregation module; and producing from the aggregation module a series of detected peaks based on detected peaks received from the analysis modules. In a further embodiment, the detected peaks produced by the aggregation module are used to determine heart rate variability/complexity. At least one embodiment includes a system for performing any of the above methods.

This invention was made with government support under Contract No. W81XWH-08-D-00170 awarded by the U.S. Army Institute of Surgical Research, Fort Sam, Houston. The government has certain rights in the invention.

### I. FIELD OF THE INVENTION

The invention in at least one embodiment relates to a fusion model using electrocardiogram leads and arterial blood pressure to determine heart-rate complexity.

### II. BACKGROUND OF THE INVENTION

Over 35 years have passed since the introduction of a real-time QRS detection algorithm by Pan and Tompkins. The QRS complex is the combination of three of the graphical deflections of a typical electrocardiogram (ECG), in particular the Q, R, and S waves. Nevertheless, the detection of QRS complexes within an ECG remains as ever an important problem and topic of research. Reliable detection implies reliable determination of the heart rate as well as other heart-related statistics, and hence, reliable detection aids diagnosis, treatment, and the saving of lives.

Real-time heart-rate complexity (HRC) analysis and/or heart-rate variability (HRV) analysis in critically ill or injured patients requires accurate, automated detection of QRS complexes. Despite the fact that numerous QRS detection algorithms now exist, accurate detection remains a practical challenge.

HRC and HRV tools are used to quantify beat-to-beat changes in the R-to-R interval (RRI), that is, the beat interval between the most prominent peaks (R waves) of the ECG and therefore reflect different physiological factors modulating normal sinus rhythm. A multicenter randomized controlled trial in very-low-birth-weight infants showed that clinical use of HRC-based monitoring in the neonatal intensive care unit may reduce mortality in infants on HRC monitoring versus those without it. Previous studies have shown that HRC and HRV not only are useful for monitoring patients in the critical care environment but may also provide an indication of the need for lifesaving interventions in patients with trauma. Because calculation of signal-derived metrics such as HRC and HRV relies on accurate R-wave detection (RWD), a hurdle in using HRC as a new vital sign is that ECG review with manual RWD has been needed, which is not practical for real-time continuous application. Therefore, widespread clinical use of ECG-based monitoring will require continuous, accurate, and automated RWD.

Unfortunately, although the biomedical research community has witnessed a plethora of viable techniques for detecting different fiducial points on the ECG, RWD remains a practical challenge in many situations. Although many published RWD algorithms have performed similarly on ECG recordings from standard databases, from past experience and testing, these algorithms have failed to perform, as well, when confronted with real-time ECG signals in an ambulatory or hospital environment. There may be several reasons for this. First, many RWD algorithms were either designed to process ECGs offline and at specific sampling rates or tested retrospectively without formal real-time verification. Second, standard ECG databases do not always provide the signal morphologies and durations—including setup times, delays, and other temporal factors experienced in the intensive care unit—needed to develop and validate these algorithms. Hence, published RWD algorithms may not have encountered signals with a wide range of morphologies (e.g., those described by hemorrhagic shock [HS] or have lost ECG characteristics) and/or different kinds of noise, be it electromechanical, organic, or human related. Lastly, many algorithms have not been adapted to handle data from currently available medical devices and cannot be of clinical use without prior knowledge and tweaking of these algorithms.

The concept of data fusion, particularly for ECG analysis, has been explored by many authors. For example, since the Computers in Cardiology Challenge, which began in 2000, Physionet has helped stimulate the use and development of eclectic methods through competitions. However, most of the approaches that were developed for these challenges, be they classifying the quality of ECGs or distinguishing between heart rhythms, did not involve multiple RWD algorithms, other than the PhysioNet/Computers in Cardiology Challenge 2006: QT Interval Measurement. In this latter case, limited details were provided for developing the algorithm “Meta-6” or describing the process. Although two RWD algorithms have been used by Li et al. for the estimation of heart-related features such as heart rate and blood pressures, their approach involves the fusion of signal quality indices, with scarce discussion on the fusion of RWD outputs and other real-time RWD issues. Likewise, many fusion strategies for RWD have focused on neural networks and empirical mode decomposition to adaptively adjust system outputs.

Heart-rate complexity (HRC) quantifies the amount of complex variability or irregularity in the heart-rate time series. It is most often obtained by analyzing the R-to-R interval (RRI) of 800 beats or more from a patient's ECG. HRC has been shown to be a sensitive marker of physiologic state during blood loss and trauma, and is associated with mortality and the need to perform life-saving interventions in trauma patients. However, ECG waveforms are often corrupted by artifacts, missing data, and noise that is non-Gaussian and non-stationary. Calculating reliable, real-time HRC values from such signals, and providing confidence intervals for the estimates, is therefore difficult. This is especially true for trauma or critically ill patients.

### III. SUMMARY OF THE INVENTION

The invention in at least one embodiment includes a system including: a plurality of medical sensors, wherein at least one medical sensor provides at least one ECG lead and at least one medical sensor provides at least one pressure reading; a plurality of analysis modules, wherein each analysis module is in communication with at least one medical sensor, each analysis module is configured to detect peaks in a signal received from the at least one medical sensor in which the analysis module is in communication; and an aggregation module in communication with each of the plurality of analysis modules, the aggregation module configured to provide a series of identified peaks representing heartbeats as an output based on detected peaks received from said plurality of analysis modules.

In a further embodiment, each analysis module includes at least three detection algorithm modules each having a pre-processing section and an analysis section, and a fusion module in communication with each of the detection algorithm modules. In a further embodiment to the first embodiment, each analysis module includes four detection algorithm modules, wherein each detection algorithm module receives as an input the signal provided by the at least one medical sensor in communication with the analysis module and each detection algorithm module configured to use different logic to detect a beat in the input signal, and a fusion module receiving an output from each of the detection algorithm modules and configured to determine which detected beat to be outputted by the analysis module. In a further embodiment to either of the previous two embodiments, at least one analysis module includes at least two detection algorithm modules that share a common pre-processing section. In a further embodiment to the other embodiments of this paragraph, the detection algorithm modules present in any one analysis module is based on a type of the signal to be received from at least one medical sensor. In a further embodiment to the other embodiments in this paragraph, at least one analysis module includes among the detection algorithm modules two detection algorithm modules having a pre-processing section configured to decompose the signal into sub-bands prior to feature extraction, and an analysis section applying a set of heuristic rules through a set of five levels. In a further embodiment to the other embodiments in this paragraph, at least one analysis module includes among the detection algorithm modules two detection algorithm modules having an analysis filter bank for receiving the input signal, wherein the analysis filter bank providing four outputs summed in a variety of combinations, a plurality of moving window integrators in communication with the analysis filter bank to receive summed outputs, a set of five processing levels in communication with the plurality of moving window integrators configured to produce at least one detected beat to the fusion module.

In a further embodiment to any of the above embodiments, the fusion module is configured to receive quantitative decisions in a buffer of the fusion module from the decision algorithm modules in communication with the fusion module; determine whether quantitative decisions have been received from all of the decision algorithm modules; determine whether a most recent received quantitative decision is a second consecutive decision from one detection algorithm module; when either determination is positive, fusing the received quantitative decisions, and determining an existence of a peak and outputting any peak that exists; and when neither determination is positive, waiting for the next quantitative decision.

In a further embodiment to any of the above embodiments, the system further includes a heart rate variability/complexity module in communication with the aggregation module and configured to receive the output of the aggregation module. In a further embodiment to any of the above embodiments, the system further includes a signal validation module in communication with the aggregation module.

The invention in at least one embodiment includes a method for providing a beat determination based on signals from a plurality of medical sensors attached to one patient in substantially real time, the method including: receiving in at least two analysis modules at least one signal from at least one medical sensor, wherein the signal includes at least one of an ECG signal and a pulse signal such that at least one analysis module receives one ECG signal and at least one analysis module receives one pulse signal; in each analysis module processing one signal through at least four detection algorithm modules to produce at least one quantitative decision, fusing the quantitative decisions together with a fusion module, and outputting at least one detected peak from the fusion module to an aggregation module; and producing from the aggregation module a series of detected peaks representing heartbeats of the patient based on detected peaks received from the analysis modules. In a further embodiment, producing the series of detected peaks is based on a beat signal quality index for each analysis module with the analysis module with the highest best signal quality index being used to supply the detected peaks for output by the aggregation module. In a further embodiment to the previous embodiment, the beat signal quality index equals the beats detected by that analysis module over a predetermined time period divided by an average of the total number of beats detected by all of the analysis modules over the predetermined time period.

In a further embodiment to the previous method embodiment, fusing the quantitative decisions together includes receiving quantitative decisions in a buffer of the fusion module from the decision algorithm modules in communication with the fusion module; determining whether quantitative decisions have been received from all of the decision algorithm modules; determining whether a most recent received quantitative decision is a second consecutive decision from one detection algorithm module; when either determination is positive, fusing the received quantitative decisions, and determining an existence of a peak and outputting any peak that exists; and when neither determination is positive, waiting for the next quantitative decision to be received from the detection algorithm modules. In a further embodiment, fusing the quantitative decisions occurs after elapse of a refractory period after the last detected peak.

In a further embodiment to the previous method embodiment, each detection algorithm module pre-processes the received signal to at least one of filter the signal and extract features from the signal to provide at least one processed signal, analyzes the at least one processed signal to detect whether a beat is present in the signal.

In a further embodiment to the previous method embodiment, aggregating includes receiving detected peaks in a buffer from the analysis modules; determining whether detected peaks have been received from all of the analysis modules; determining whether a most recent received detected peak is a second consecutive detected peak from one analysis module; when either determination is positive, fusing the received detected peaks, and determining an existence of a peak and outputting any peak that exists; and when neither determination is positive, waiting for the next detected peak to be received from the analysis modules. In a further embodiment to the previous embodiment, fusing the received detected peaks includes analyzing the detected peaks using centroid analysis to detect output agreement. In a further embodiment to the previous embodiment, output agreement is based on K-nearest neighbor.

In a further embodiment to the previous method embodiments, the method includes receiving the series of detected peaks from the aggregation module into a heart rate complexity module, determining a heart rate complexity by the heart rate complexity module based on the received detected peaks, and triggering an alarm when the heart rate complexity is outside a predetermined range.

The invention in at least one embodiment includes a computer program product claim for detecting peaks in a plurality of biomedical signals, the computer program product including: a computer readable storage medium having encoded thereon: first program instructions executable by a processor to cause the processer to receive a plurality of biomedical signals originating from a plurality of medical sensors; second program instructions executable by a processor to cause the processer to assign each signal to a particular analysis module based on a type of signal that the signal is; third program instructions executable by a processor to cause the processer to use each analysis module to analyze one signal with a plurality of algorithms; fourth program instructions executable by a processor to cause the processer to use each analysis module to fuse the outputs of the plurality of algorithms to provide an output; fifth program instructions executable by a processor to cause the processer to select the highest quality output from the plurality of analysis modules; and sixth program instructions executable by a processor to cause the processer to use the selected output to determine at least one of heart rate complexity and heart rate variability.

The purpose of at least one embodiment of the invention is to provide a reliable method for determining a continuous value of HRC in real time for critical care patients using multiple waveform channels that also compensates for noise and the unreliability of data. The method is based upon the concept of fusing detected peak-to-peak interval (PPI) and RRI estimates derived from multiple noisy waveform sources, such as from arterial blood pressures (ABP) and multiple ECG leads, during intensive care unit (ICU) monitoring. This approach in at least one embodiment automatically rejects degraded waveform data. It is hypothesized that a recursive fusion of outputs—first, from several best, published peak detection (PD) algorithms; then, from multiple noisy waveform channels and derived SQIs—could produce a more robust real-time solution for calculating HRC in critically ill patients. By practically fusing the outputs of multiple real-time PD algorithms on multiple waveforms to calculate a streaming HRC value, the data fusion framework may be easily integrated into a real-time HRC software program for decision support and triage in critically ill patients.

The invention in at least one embodiment includes a method for calculating heart-rate variability measures using multiple waveform sources. Calculation of these measures can be performed along with HRC calculations.

The invention in at least one embodiment includes a method for calculating heart-rate complexity measures using multiple waveform sources. In a further embodiment, the invention includes a method for calculating heart-rate variability measures using multiple waveform sources.

### IV. BRIEF DESCRIPTION OF THE DRAWINGS

FIG. 1 illustrates an operational overview of an example embodiment according to the invention.

FIG. 2 illustrates a method according to at least one embodiment of the invention.

FIGS. 3A and 3B illustrate systems according to at least one embodiment of the invention.

FIGS. 4A and 4B illustrate example analysis modules according to at least one embodiment of the invention.

FIGS. 5A-5G illustrate example detection algorithm modules according to at least one embodiment of the invention.

FIG. 6 illustrates a method according to at least one embodiment of the invention.

FIG. 7 illustrates a system according to at least one embodiment of the invention.

FIGS. 8A-8F depict histogram plots for R-R intervals with FIG. 8A depicting manual determinations, FIG. 8B depicting fusion, FIG. 8C depicting ECG Lead I, FIG. 8D depicting ECG Lead II, FIG. 8E depicting ECG Lead IV, and FIG. 8F depicting ABP.

FIG. 9 illustrates a computer program product and computer implementation according to an embodiment of the invention.

### V. DETAILED DESCRIPTION OF THE DRAWINGS

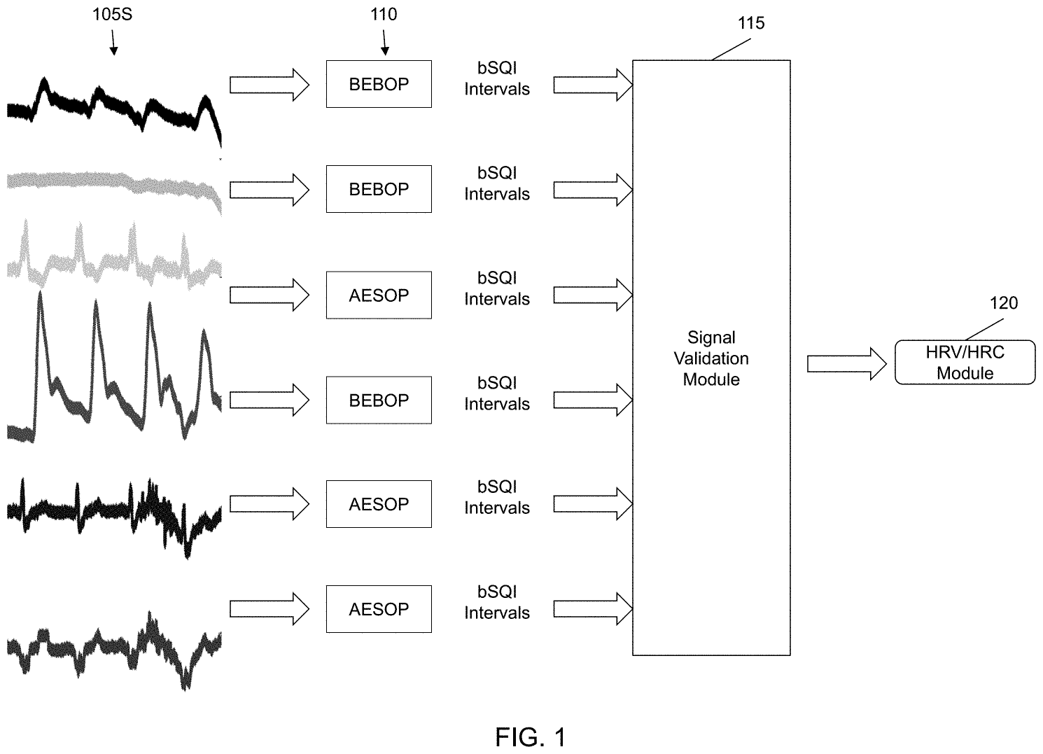

In at least one embodiment, the invention includes a method for analyzing signal data from one or more medical sensors measuring, for example, ECG; pulse oximeter; pressure sensors providing blood pressure, pulmonary arterial pressure, central venous pressure, arterial pressure; or blood oxygenation (saturation of peripheral oxygen, SpO2) to detect a patient's peak-to-peak interval (PPI) sequence automatically and substantially in real-time. FIG. 1 illustrates an overview of the operation of an example embodiment according to the invention. The system receives a plurality of signals 105S from a plurality of medical sensors with the signals being processed by analysis modules (AESOP (Automated Electrocardiogram Selection of Peaks) and BEBOP (Bypassing ECG Beats or Peaks)) 110 whose outputs (bSQI values and Intervals) are scored by a signal validation module 115 for quality prior to validation and selection of a signal to provide a HRV/HRC system (or module) 120.

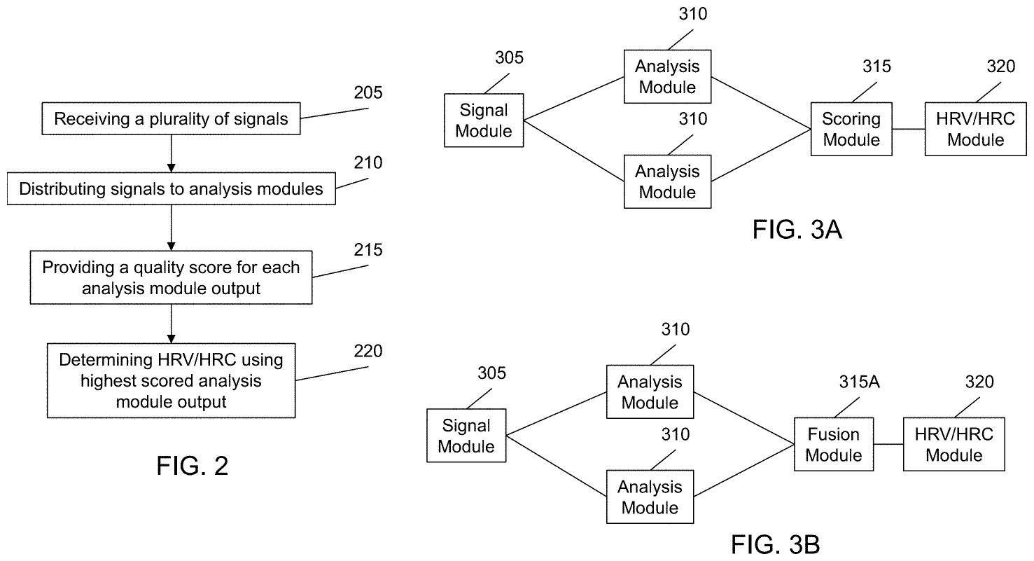

FIG. 2 illustrates such a method. The system receives a plurality of output signals from at least one medical sensor, 205. The signals are distributed to a plurality of analysis modules for processing and outputting of a detecting heart rate and/or PPI, 210. The outputs of multiple modules are analyzed by a scorer module that provides a quality score for each output, 215. The system then uses the analysis module output with the highest quality score to determine the HRV/HRC in the HRV/HRC module, 220; or alternatively to output the highest quality score analysis module output. In a further embodiment, each analysis module includes a plurality of detection algorithms that are fused together to provide an output. In a further embodiment, there are four detection algorithm modules present in each analysis module.

In at least one embodiment, the HRV/HRC module 120 illustrated in FIG. 1 and similar modules 320 illustrated in FIGS. 3A and 3B determine variability and/or complexity based on at least one metric identified in Table 1. For example, sliding windows of 200 RRIs are employed for short-term variability/complexity measures, and 800 RRIs, for long-term variability/complexity measures. In at least one embodiment, the HRV/HRC module 120 illustrated in FIG. 1 and similar modules 320 illustrated in FIGS. 3A and 3B employ values of m=2, r=6, and s=2, 3, and 4 according to the Appendix.

TABLE 1

| Measure | Notation | Type of Analysis |
|---|---|---|
| Sample Entropy | SampEn | Short-term complexity |
| Quadratic | QSE | Short-term complexity |
| Sample Entropy | | |
| Multisource | MSE | Long-term complexity |
| Entropy | | |
| Poincaré | SD1/SD2 | Short-term variability |
| Variability Ratio | | |
| Fractal Scaling | α | Long-term variability |
| Exponent | | |
| Autocorrelation | A(k, τ) | Long-term variability |
| Coefficient | | |
| Degree of | StatAv | Long-term variability |
| Nonstationarity | | |

In at least one embodiment, the nature or type of signal determines which analysis module receives it. For example, ECG leads I, II, and IV may be distributed to one type of an analysis module, while signals relating to pulmonary arterial pressure, central venous pressure, arterial pressure, and blood oxygenation may be distributed to a different type of an analysis module similar to the AESOP and BEBOP modules illustrated in FIG. 1.

FIG. 3A illustrates a system embodiment according to the invention. The illustrated system includes a signals module 305, at least two analysis modules 310, a scorer module 315, and an optional HRV/HRC module 320. In an alternative embodiment, the scorer module 315 is replaced by a fusion module 315A as illustrated in FIG. 3B. In at least one embodiment, the scorer module and the fusion module are examples of a validation module. In an alternative embodiment, there is one analysis module and the scorer module is omitted.

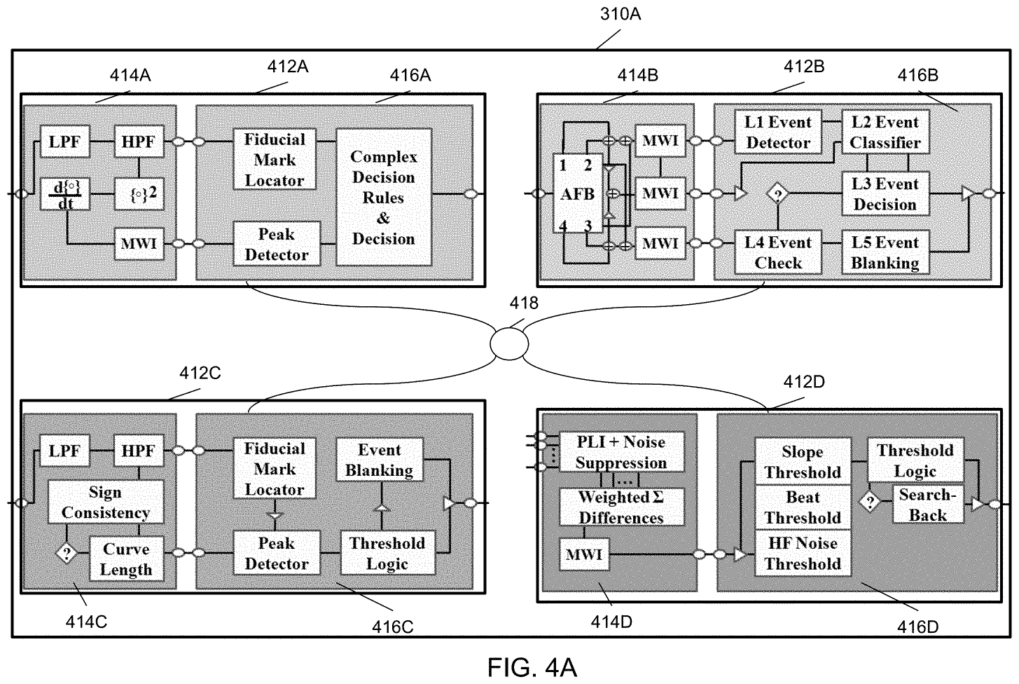

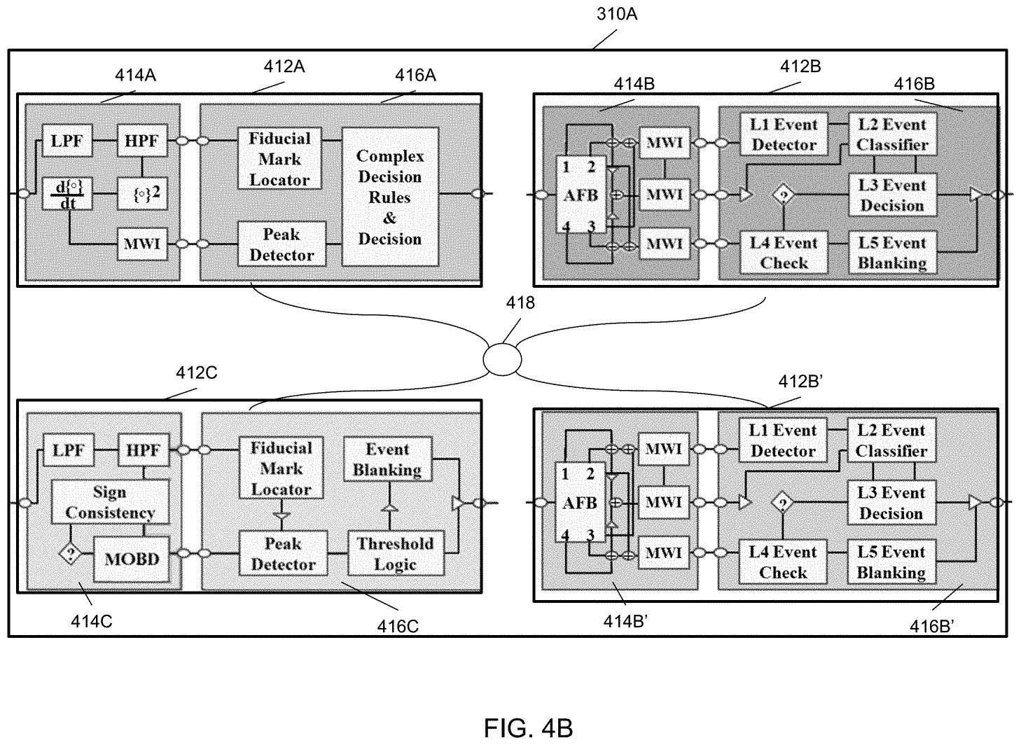

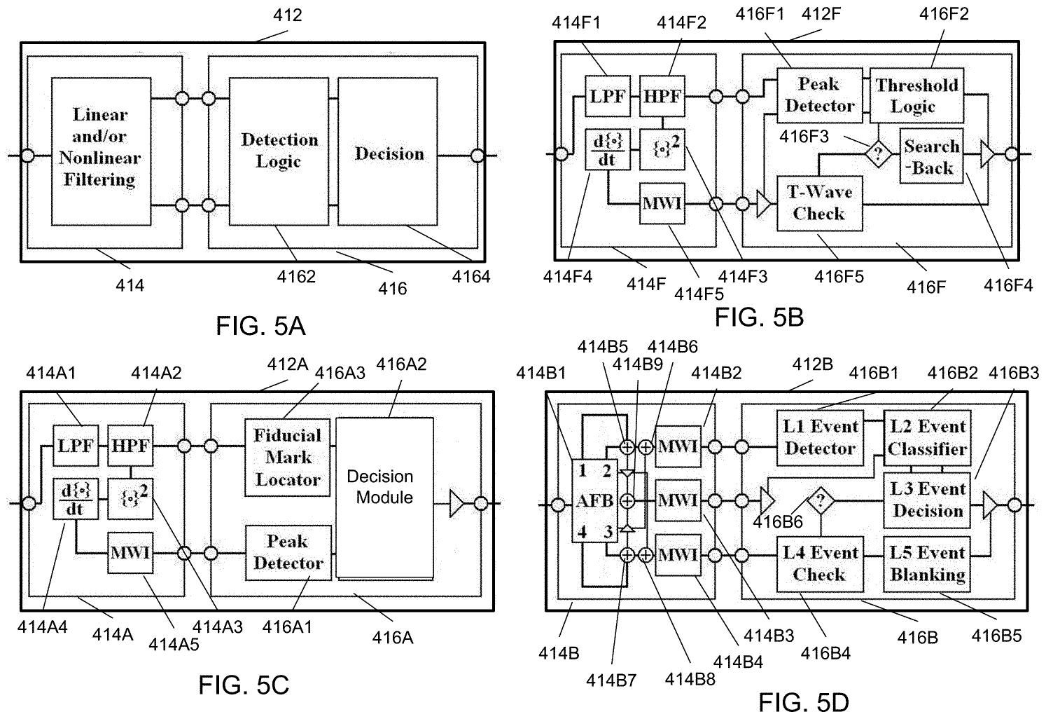

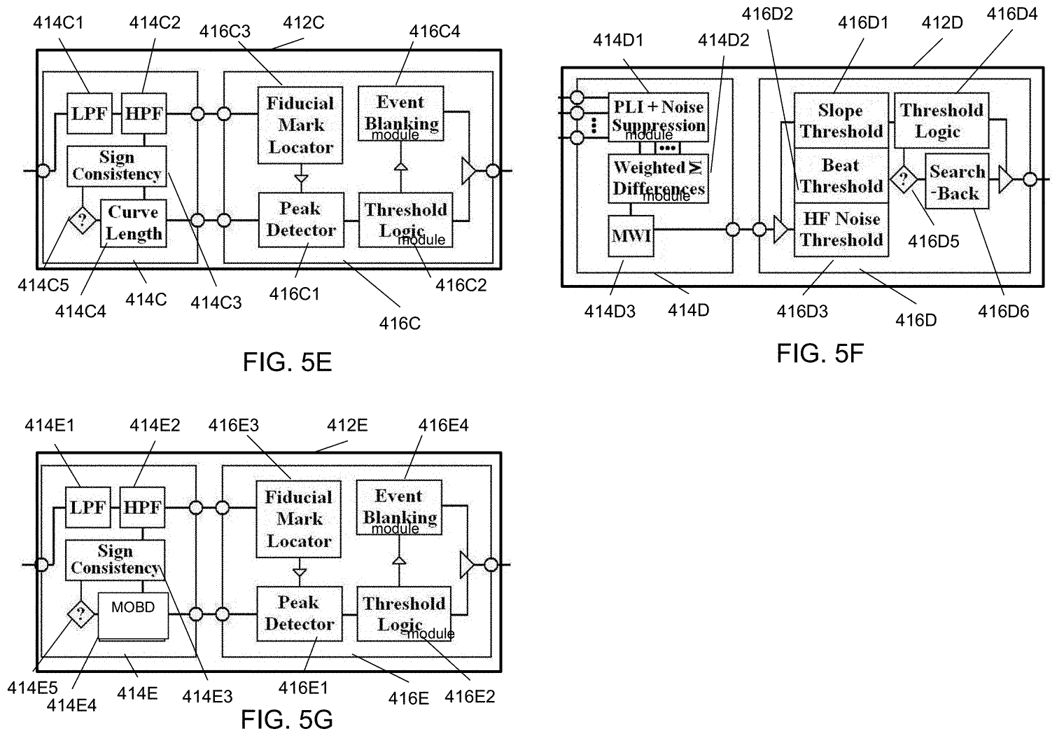

FIG. 4A illustrates an example of an analysis module 310A having a plurality of detection algorithm modules 412A, 412B, 412C, 412D, which receive the input into the analysis module 310A, connected to a fusion module 418 that provides the output of the analysis module. In at least one embodiment, each detection algorithm module includes a pre-processing section 414A, 414B, 414C, 414D and an analysis section 416A, 416B, 416C, 416D. In an alternative embodiment, where two or more detection algorithm modules have a common pre-processing section in a particular analysis module, then these detection algorithm modules share a common pre-processing section that outputs then to the different analysis sections. FIG. 4B illustrates another example of an analysis module 310B where two detection algorithm modules 412B and 412B′ (e.g., in place of detection algorithm module 412D in FIG. 4A) have similar components that in at least one embodiment use different parameters as discussed later in connection with FIG. 5D.

In a further embodiment, there are at least two types of analysis modules present in the system that are selected for a particular signal based on the type of signal to be analyzed as discussed previously. In at least one embodiment, there are an automated electrocardiogram selection of peaks (AESOP) analysis module and a bypassing ECG beats or peaks (BEBOP) analysis module. In at least one embodiment where there are at least two different types of analysis modules, at least two types of analysis modules share at least one common detection algorithm module.

FIGS. 5A-5G illustrate examples of detection algorithm modules that may be used as part of the system. Each of the illustrated examples find inspiration from different existing methodologies for detecting beats, which are largely done after the fact as opposed to substantially in real-time as occurs in at least one embodiment. Based on this disclosure, one of ordinary skill in the art will appreciate that other detection algorithm modules may be used to detect peaks in signals such that the detected peaks are representative of heartbeats.

Although the detection algorithm modules illustrated in FIGS. 5A-5G are discussed in terms of ECG signals, based on this disclosure one of ordinary skill in the art will appreciate that there are other medical signals that share similar traits to an ECG signal and as such may be used as the input instead.

FIG. 5A illustrates a general detection algorithm module 412 that illustrates the common elements shared by detection algorithm modules for use in at least one embodiment. Detection algorithm modules 412 detect the QRS complexes in an ECG signal by using, for example, algorithms that provide beat detection including approaches that rely on more specific waveforms and/or fiducial points such as P waves, R waves, and T waves. The pre-processing section 414, which is illustrated as linear and/or nonlinear filtering in FIG. 5A, provides pre-processing or feature extraction that filters an ECG signal for use by the analysis section 416, which is illustrated as having detection logic and decision components 4162, 4164. As such the pre-processing section 414 prepares the ECG signal for processing by the detection algorithm module 412. The detection logic and decision components 4162, 4164 in at least one embodiment are combined into one detection component (or analysis section 416). Examples of the processing that may be used by the detection logic and decision components 4162, 4164 include, but are not limited to, derivative-based filters accompanied by adaptive thresholds, filter banks, wavelets and singularity detection, neural networks and frequency modeling, non-linear transforms, and linear prediction.

FIG. 5B illustrates a detection algorithm module 412F based on at least one article by Pan and Tompkins. The illustrated module is an example of a threshold-based QRS detection module. The detection algorithm module 412F uses a mixture of derivative-based signal processing, integer filters, the adaption of thresholds using recent signal peaks and noise peaks, a search-back mechanism for finding missed beats, refractory blanking, and T-wave identification.

The illustrated pre-processing section 414F includes a cascaded low-pass filter (LPF) 414F1 followed by high-pass filter (HPF) 414F2 that combined attenuate the noise to improve the input's signal-to-noise ratio (SNR). Based on this disclosure, one of ordinary skill in the art should appreciate that the filter order may be switched or other approaches may be used to provide the cascade filtering functionality. In at least one embodiment, the cascaded filters lower the required thresholds for increased detection sensitivity that leads to reduced false detections caused by artifacts. The output from the cascaded filters is squared by a squarer module 414F3 in at least one embodiment to intensify the differentiated input's frequency response slope and emphasize signal frequencies leading to a reduction in false detections caused by T waves. The signal is then differentiated by the differentiator 414F4 in at least one embodiment to obtain QRS-complex slope information, which will later be used to determine when a rising edge of the final pre-processed inputs occurs and can be equivalent to a candidate QRS event. The signal is then integrated with a moving window integrator (MWI) 414F5 in order to obtain R-wave slope information and a QRS-complex width for threshold comparisons. In at least one embodiment, there are two outputs from the pre-processing section 414A: the high pass filter 414F2 and the moving window integrator 414F5.

A peak detector 416F1 in the analysis section 416F receives an output from the high pass filter 414F2 and the MWI 414F5 for detection of a peak by comparing the two signals against two respective sets of thresholds: signal and noise peak thresholds from the threshold logic 416F2. When both comparisons pass, the decision component 416F3 confirms that a QRS complex has occurred, and then the decision component 416F3 adjusts these thresholds. In at least one embodiment, the algorithm uses a dual-threshold technique to find missed beats, and thereby reduce false negatives. There are two separate threshold levels in each of the two sets of the thresholds. One level is half of the other level. The thresholds continuously adapt to the characteristics of the signal since they are based upon the most-recent signal and noise peaks that are detected in the ongoing processed signals. If the program does not find a QRS complex in the time interval corresponding to 166 percent of the current average RR interval, the maximal peak detected in that time interval that lies between these two thresholds is considered to be a possible QRS complex, and the lower of the two thresholds is applied. In at least one embodiment, this avoids requiring a long memory buffer to store the past history of the ECG, and thus require minimal computing time to accomplish the search-back procedure to look for a missing QRS complex.

In at least one embodiment, where the heart rate is irregular such as bigeminy or trigemini, the missed beat cannot be found by searching back through the same time interval as for regular heart rates. For the case of irregular heart rates, both thresholds are reduced by half in order to increase the sensitivity of detection and to avoid missing valid beats.

In a further embodiment, the search-back component 416F4 uses percentages. In at least one embodiment, the detection logic component maintains two average R-R intervals in order to handle missed beats as well as irregular heartbeats. The decision component 416F3 in at least one embodiment will disregard a found QRS complex when the T-Wave Check module 416F5 determines that the peak belongs to a T wave.

FIG. 5C illustrates a detection algorithm module 412A based on at least one article by Hamilton and Tompkins. This detection algorithm module 412A shares a similar pre-processing section 414F with the module illustrated in FIG. 5B. In at least one alternative embodiment, the detection algorithm module 412A is implemented with slightly different difference equations in the pre-processing section 414A.

The analysis section 416A in at least one embodiment utilize the approaches of the previous module, but also fiducial mark placement and consistency, mean peak level estimation, baseline shift discrimination, and optimization of search-back detection thresholds. These additional capabilities, excluding fiducial mark placement, are encapsulated in the decision module 416A2. In at least one embodiment, the peak detector 416A1 receives its input from the MWI 414A5. The peak detector 416A1 detects a local maximum/peak when the slope level drops below half the distance from the maximal value to the base point or once a predetermined time, which in at least one embodiment is above 175 ms, has elapsed after a maximal positive slope in the integrated signal from the MWI 414A5. The detected peak is passed to the decision module 416A2, which in at least one embodiment incorporates similar components as the threshold logic 416F2, decision component 416F3, search-back component 416F4, and T-Wave Check module 416F5 illustrated in FIG. 5B. In at least one embodiment, the module illustrated in FIG. 5C has undergone further threshold optimization than the modules illustrated in FIG. 5B prior to implementation.

In at least one embodiment, a fiducial mark locater 416A3 is used to mark the largest peak in the filtered signal outputted by the high-pass filter 414A2 (or alternatively a set of cascade filters). In at least one embodiment, the filtered signal is in a window of 225 ms to 125 ms preceding a peak detection in the MWI signal or between 250 ms and 150 ms for T waves.

In at least one alternative embodiment, when a missing beat is likely to have occurred, the decision component calculates a search-back detection threshold in order to find missing beats. In a further alternative embodiment, the decision component 416A2 performs refractory blanking, the identification of T waves, and a baseline shift discrimination to locate a missing beat.

In at least one embodiment, once a valid QRS complex is recognized, there is a 200 ms refractory period before the next one can be detected since QRS complexes cannot occur more closely than this physiologically. This refractory period eliminates the possibility of a false detection such as multiple triggering on the same QRS complex during this time interval. When a QRS detection occurs following the end of the refractory period but within 360 ms of the previous complex, it should be determined if it is a valid QRS complex or a T wave. In this case, the waveform with the largest slope is judged to be the QRS complex.

To be reliable, a QRS detection algorithm should adapt each of its parameters with time so as to be able to operate properly for ECG's of different patients as well as for ECG morphology changes in a single patient. In at least one embodiment, each threshold automatically adapts periodically based upon peak values of signal and noise. When a QRS must be found using second (lower) thresholds, threshold readjustment occurs twice as fast as usual. In the dual-threshold technique, the RR-interval average must be updated for each heartbeat.

In at least one embodiment, two separate measurements of the average RR-interval are maintained. One RR-interval average is the mean of all of the most recent eight RR-intervals. A second RR-interval average is the mean of the most recent eight beats that fell within the range of 92-116 percent of the current RR-interval average. Without this first average, this approach would be suitable only for a slowly changing and regular heart rate. In at least one embodiment when the heart rate suddenly changes, the first RR-interval average substitutes for the second one allowing the algorithm to adapt rapidly to a changing signal. In at least one embodiment when another patient's ECG is inputted into the system, it can adapt rapidly without requiring special learning phases in part due to the two RR-interval averages.

FIG. 5D illustrates a detection algorithm module based on at least one article by Afonso, Tompkins, Nguyen and Luo. The illustrated detection algorithm module 412B is based on sub-band decomposition before feature extraction occurs to provide a set of signals to a series of decision levels that apply heuristic rules at each level to detect a beat in the signal. In at least one embodiment, the approach includes at least one of the following: multi-rate signal processing and feature extraction, signal decomposition into sub-bands using a filter bank, single-channel detection blocks and decision levels, the adaptation of detection strengths using signal and noise histories, and partial refractory blanking.

The illustrated pre-processing section 414B includes an analysis filter bank (AFB) 41461; a plurality of MWIs 414B2, 414B3, 414B4; and a plurality of summers (or summation modules) 414B5, 414B6, 414B7, 414B8, 414B9. In at least one embodiment, the input signal is decomposed by the AFB 414B1 into four relatively uniformly wide sub-bands occupying a frequency band, which in at least one embodiment is 5.6 Hz to 28.1 Hz. The AFB 414B1 in at least one embodiment down samples and band-pass filters the input with a parallel set of analysis filters using a polyphase implementation. In at least one embodiment, this implementation lowers the processing rate and captures specific energy distributions. In an alternative embodiment, the up sample and reconstruction of the input is performed with a polyphase implementation of a synthesis filter bank in place of the AFB 41461.

The illustrated embodiment includes the AFB 414B1 providing four outputs 1, 2, 3, and 4 that are provided in summation with other outputs to three MWIs 414B2, 414B3, 414B4. Outputs 1 and 2 are summed together by summer 414B5 to provide the summation to be summed with output 3 by summer 414B6 to provide the input to a first MWI 414B2. The summation of outputs 1 and 2 is summed by summer 414B9 with the summation of outputs 3 and 4 from summer 414B7 to provide the input into the second MWI 414B3. The summation of outputs 3 and 4 by summer 414B7 is summed with output 2 by summer 414B8 to provide the input into the third MWI 414B4. In at least one embodiment, the summations provide for combining the energy distributions across multiple sub-bands. The three MWIs 414B2, 414B3, 414B4 output a respective first feature, second feature and third feature to the analysis section 416B.

The illustrated analysis section 416B includes five event elements (or levels) 416B1, 416B2, 416B3, 416B4, 416B5 that receive a variety of outputs from at least one MWI and other elements. The L1 event detector 416E31 receives the first feature from the first MWI 414B2. The L1 event detector 416E31 determines when the first feature changes direction within a given time interval to detect a peak/inflection point. The L2 classifier 416B2 compares two detection strengths derived from the detected peak of the first feature on the second feature, which is received from the second MWI 414B3, and respective single-channel signal/noise histories against two thresholds to classify the peak twice. Similar to the Pan-Tompkins and Hamilton-Tompkins algorithms, the source of the histories and thresholds is the ECG waveform data. The L2 event classifier 416B2 then updates all of the histories.

The L3 event decision module 416B3 after the histories are updated fuses the two classifications in order to produce an event decision. This module 416B3 fuses the beat detection status from each of the 2 one-channel detection algorithms in the L2 classifier 416B2 by incorporating a set of if-then-else rules. The rules incorporate the 2 one-channel detection blocks have complementary detection rates. There are four possible cases to design rules in at least one embodiment. If both channels indicate a beat then the output of the L3 event decision module 416B3 classifies the current event as a beat. Since channel 2 uses a high threshold in its detection logic, it generates few false positives (FP's) and, thus, beat detection is very accurate. If both channels indicate not-a-beat then the output of the L3 event decision module 416B3 is that the current event is not a beat. Since the threshold used in channel 1 is very low, it has very few false negatives (FN), and more than likely a beat did not occur in reality. If channel 1 indicates not-a-beat and channel 2 indicates a beat, then the output is classified as a beat. However, this scenario does not occur in at least one embodiment when the threshold used in channel 1 is very low and the same feature is used in channel 2. A beat detected by channel 2 more than likely got detected in channel 1. If channel 1 indicates a beat and channel 2 indicates not-a-beat, then the detection strengths from each channel are compared. Channel 1 generates many FP's and channel 2 generates many FN's. The normalized detection strength which indicates which decision was “stronger” can be compared to favor the channel with the stronger decision.

The L4 event check module 416B4 in at least one embodiment finds missing beats by comparing a new detection strength derived from a peak of the third feature and signal/noises histories against a one single-channel threshold. In at least one embodiment, the source of the histories and thresholds is the ECG waveform data.

The L5 event blanking module 416B5 includes a partial refractory blanking mechanism to reclassify a positive prior decision to not-a-beat if the peak on the third feature meets time/strength conditions. The L5 event blanking module 416B5 includes decision logic to eliminate possible false detection during the refractory period. In at least one embodiment, this is not a complete blanking of a beat during the refractory period, but rather a partial blanking. If a beat was detected during the refractory period (with reference to the previous beat detection) and also had a minimal detection strength in the L4 event check module 416B4 then the status of the event is changed from a beat to not-a-beat. Selection module 416B6 selects information (the third feature and histories) from the L3 event decision module 416B3 and passes them to the L4 event check module 416B4.

The detection algorithm modules illustrated in FIG. 4B that are similar to the module illustrated in FIG. 5D are directed at detecting different slope deflections. One is for detecting only positive-negative slope deflections in order to focus on systole dynamics while the other is for detecting positive-negative slope deflections of a first-order derivative of the original signal. Although the modules look the same as in FIG. 5D, whereas the module in FIG. 5D calculates absolute differences (|curr−prev|), the module 412B calculates positive differences (curr−preV) and 412B′ calculates positive differences only after differentiation of the signal.

FIG. 5E illustrates a detection algorithm module 412C based on at least one article by Zong, Moody, and Jiang. In at least one embodiment, the illustrated detection algorithm module 412C uses a combination of curve length transformation, noise suppression using sign consistency, threshold adaptation, and refractory blanking. The received signal passes through cascaded low pass filter 414C1 and high pass filter 414C2 in the pre-processing section 414C. In at least one alternative embodiment, the order the filters is reversed. The output of the cascaded filters is provided to a sign consistency module 414C3 to check for sign consistency among ordered sets of filtered inputs signals in order to suppress noise effects in the ECG signal and in at least one embodiment reduce the false detections caused by artifacts. A curve length module 414C4 transforms the filtered inputs for threshold comparison and beat detection. One example formula for this computation is:

$${L}_{n} ( w , i ) = {\sum }_{k = i - w}^{i} \sqrt{{\sum }_{j = 1}^{n} ( C + Δ {y}_{j , k}^{3} )}$$

In at least one embodiment, there is also a zeroing module 414C5 that produces a zero value in the circumstance where there is not sign consistency as determined by the sign consistency module 414C3. Although illustrated in FIG. 5E, the zeroing module 414C5 may be omitted.

The analysis section 416C in at least one embodiment utilize the approaches of the previous modules, but also fiducial mark placement and consistency, mean peak level estimation, baseline shift discrimination, and optimization of search-back detection thresholds. In at least one embodiment, the peak detector 416C1 receives its input from the curve length module 414C4. The peak detector 416C1 detects a local maximum/peak when the slope level drops below half the distance from the maximal value to the base point or once a predetermined time, which in at least one embodiment is above 175 ms, has elapsed after a maximal positive slope in the integrated signal from the MWI. The detected peak is compared by the threshold logic module 416C2 against a threshold derived from the mean signal/noise peak estimates of the integrated signal and a best coefficient and the thresholds are adjusted.

In at least one embodiment, a fiducial mark locater 416C3 is used to mark the largest peak in the filtered signal outputted by the high-pass filter 414C4 (or alternatively the cascade filter). In at least one embodiment, the filtered signal is in a window of 225 ms to 125 ms preceding a peak detection or between 250 ms and 150 ms for T waves.

The event blanking module 416C4 in the illustrated analysis section 416C is a complete blanking of a beat during the refractory period. In other words, event blocking eliminates possible false detections during the refractory period.

FIG. 5F illustrates a detection algorithm module 412D based on at least one article by Christov. In at least one embodiment, the illustrated detection algorithm module 412D uses a combination of three independent adaptive thresholds and a search-back mechanism for finding missed beats. The pre-processing section 414D includes a suppression module 414D1, a weighted differences module 414D2 and a MWI module 414D3. The suppression module 414D1 receives a single lead input with a MWI to be integrated in order to suppress power-line interference (PLI) and then each single-lead input signal is integrated with a MWI in order to suppress electromyogram and similar high-frequency noise. The weighted differences module 414D2 receives the set of integrated outputs from the suppression module 414D1. The weighted differences module 414D2 computes a weighted sum-of-difference transformation of the integrated inputs. One example formula for this computation is:

$${y}_{N} [ n ] = \frac{1}{L} {\sum }_{l = 1}^{N} \vert {x}_{l} [ n + 1 ] - {x}_{l} [ n - 1 ] \vert$$

The weighted differences module 414D2 then integrates the differentiated and averaged output with a MWI 414D3, which in at least one embodiment suppresses noise in the output due to differentiation.

The analysis section 416D compares each output to three independent adaptive thresholds in a slope threshold module 416D1, a beat threshold module 416D2, and high-frequency noise threshold module 416D3. The slope threshold provides a general detection of QRS steep slopes while the other two thresholds act as countermeasures against artifacts or high-frequency noise and low amplitude beats, respectively. These three modules 416D1, 416D2, 416D3 provide their outputs to a threshold logic module 416D4 for a determination of a peak by the decision module 416D5. In an alternative embodiment, a search-back module 416D6 is added to handle missed beats in an interval after the last detected R-R interval by maintaining one average R-R interval consisting of a set of the most recent beats, which in one embodiment is five beats.

FIG. 5G illustrates a detection algorithm module 412E based on at least one article by Suppappola and Sun. The illustrated detection algorithm module 412E transforms a band-limited ECG signal into a set of new values for comparison against an adaptive threshold. In at least one embodiment, the illustrated module uses one or more of the following: a multiplication-of-backward-difference (MOBD) transformation, a noise suppression using sign consistency, an adaption of thresholds using maximal MOBD values, and refractory blanking. The received signal passes through cascaded low pass filter 414E1 and high pass filter 414E2 in the pre-processing section 414E. The output of the cascaded filters is provided to a sign consistency module 414E3 to check for sign consistency among ordered sets of filtered inputs signals in order to suppress noise effects in the ECG signal and in at least one embodiment reduce the false detections caused by artifacts. A Nth-order MOBD module 414E4 transforms the filtered inputs for threshold comparison and beat detection. In at least one embodiment, the MOBD module 414E4 uses the following equation:

$${y}_{N} [ n ] = {\prod }_{k = 0}^{N} \vert x [ n - k ] - x [ n - k - 1 ] \vert$$

In at least one embodiment, there is also a zeroing module 414E5 that produces a zero value in the circumstance where there is not sign consistency as determined by the sign consistency module 414E3. Although illustrated in FIG. 5G, the zeroing module 414E5 may be omitted.

The analysis section 416E in at least one embodiment utilizes the approaches of the previous modules, but also fiducial mark placement and consistency, mean peak level estimation, baseline shift discrimination, and optimization of search-back detection thresholds. In at least one embodiment, the peak detector 416E1 receives its input from the curve length module 414E4. The peak detector 416E1 detects a local maximum/peak when the slope level drops below half the distance from the maximal value to the base point or once a predetermined time, which in at least one embodiment is above 175 ms, has elapsed after a maximal positive slope in the integrated signal from the MWI. The detected peak is compared by the threshold logic module 416E2 against a threshold derived from the mean signal/noise peak estimates of the integrated signal and a best coefficient and the thresholds are adjusted.

In at least one embodiment, a fiducial mark locater 416E3 is used to mark the largest peak in the filtered signal outputted by the high-pass filter 414E4 (or alternatively the cascade filter). In at least one embodiment, the filtered signal is in a window of 225 ms to 125 ms preceding a peak detection or between 250 ms and 150 ms for T waves.

The event blanking module 416E4 in the illustrated analysis section 416E provides a complete blanking of a beat during the refractory period. In other words, event blocking eliminates possible false detections during the refractory period.

In at least one embodiment, the fusion module 418 monitors the outputs from the plurality of detection algorithm modules 412 connected to it to determine when a R peak occurs based on the received outputs. In at least one embodiment, when two or more of the outputs are within close proximity to each other, the fusion module 418 determines that the R peak has occurred. In another embodiment, the output agreement occurs when at least three outputs are in agreement. In at least one further embodiment, the closeness of the outputs is less than 5 milliseconds; in a still further embodiment the time separation is less than 4 milliseconds. If the R peak is not detected within a predetermined time period to select the output that indicates a R peak closest to a running interval. In at least one embodiment, the running interval is based on the previous detected intervals, for example, the last five, seven, or fifteen intervals detected by the fusion module. Further examples of the number of intervals includes any integer number less than 10, any integer number less than 30, or any integer number. In an alternative embodiment, the fusion module uses the centroid analysis to detect output agreement based, for example, on the K-nearest neighbor (KNN) algorithm although other approaches may be used.

In a further embodiment, the fusion module 418 determines when agreement is present between the detection algorithm modules.

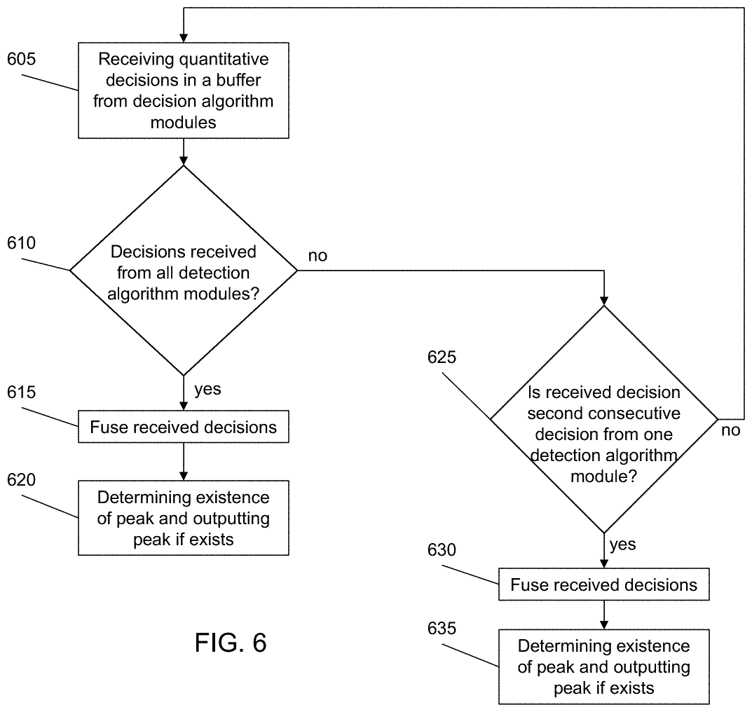

The method illustrated in FIG. 6 provides an example of how the fusion module 418 may receive the outputs of the detection algorithm modules. Each quantitative decision provided by the detection algorithm modules will exceed a minimum threshold set based on the refractory period, which in at least one embodiment is defined as 240 milliseconds. For a quantitative decision to be considered, the time difference between it and the previous quantitative decision from that detection algorithm module will need to exceed the minimum threshold. Otherwise the quantitative decision will be disregarded by at least one of the detection algorithm module or the fusion module. The quantitative decisions are received by an input buffer in the fusion module, 605, to allow for a set to be accumulated such that if each detection algorithm module provides a respective quantitative decision before any one of the detection algorithm modules provides two consecutive quantitative decisions, 610, then all of the quantitative decisions in the input buffer are fused together, 615, and a determination is made for the current time frame, 620.

However, if the input buffer receives two consecutive quantitative decisions from one detection algorithm module before receiving quantitative decisions from all of the other detection algorithm modules for the current time period, 625, then the received quantitative decisions before the consecutive quantitative decision are fused together, 630, and a determination is made for the current time frame, 635.

During fusion, the fusion module determines whether all outputs (peak-to-peak intervals) are equal, and if so, then a beat is detected and outputted by the fusion module. Otherwise, no beat is outputted by the fusion module.

In an alternative embodiment, the fusion module weighs other factors when not all individual outputs are equal. Define a mode as the same output reported by at least two component algorithms. If there are two modes (i.e., two different pairs of detection algorithm modules agreed), the first mode is outputted. If there is one mode, it is outputted. If there are no modes and multiple algorithms reported a beat, the output interval closest to the average of the previous twelve final outputs is returned as the new final output. In alternative embodiments, the previous five, seven, ten, or fifteen final outputs are used instead of the previous twelve final outputs. Otherwise, no output is provided by the fusion module.

In an alternative embodiment, the fusion module records a plurality of quantitative decisions from the detection algorithm modules over a period of time to allow for one or more detection algorithm modules to detect a missing beat in their respective outputs. In at least one embodiment, the buffer would hold readings over a predetermined time or a predetermined number of readings prior to analyzing the earliest quantitative decisions to produce an output to the scoring module 315. In a further embodiment, the buffer would operate on a first-in-first-out for handling the quantitative decisions. In such an embodiment, each of the analysis modules would have the same processing delay.

In at least one embodiment, the scoring module 315 scores each of the outputs received from the connected analysis modules 310. In at least one embodiment, the scoring is based on the number of detected beats over a predetermined time and the number of actual beats over that same predetermined time. The predefined time is a predefined beat window that is used. The score for any one analysis module in at least one embodiment is the number of detected beats in the beat window divided by the number of actual beats in that beat window. In at least one embodiment, the number of actual beats is the average of the number of beats detected by all of the analysis modules in the system, in a further embodiment, the average is taken from all of the analysis modules by removing at least one of the lowest detected beat totals from the average, and in an alternative embodiment at least one of the highest detected beat totals is removed from the average. The analysis module 310 with the highest score is used to provide the output to the HRV/HRC module 320 or as an output from the system. In at least one embodiment, the analysis module 310 selected can change from beat to beat or alternatively after a use window has expired before scoring occurs again where the use window is a predefined time period or a number of beats having been detected by the selected analysis module or the system depending on the particular implementation.

In an alternative embodiment illustrated in FIG. 3B, the fusion module 315A takes the place of the scoring module 315. In at least one embodiment, the fusion module 315A monitors the outputs from the plurality of analysis modules 310 connected to it to determine when a R peak occurs based on the received outputs. In at least one embodiment, when a predetermined number of outputs (e.g., 2, 3, or 4) are within close proximity to each other, the fusion module 315A determines that the R peak has occurred. In at least one further embodiment, the closeness of the outputs is less than 5 milliseconds; in a still further embodiment the time separation is less than 4 milliseconds. If the R peak is not detected within a predetermined time period to select the output that indicates a R peak closest to a running interval. In at least one embodiment, the running interval is based on the previous detected intervals, for example, the last five, seven, or fifteen intervals detected by the fusion module. Further examples of the number of intervals includes any integer number less than 10, any integer number less than 30, or any integer number. In an alternative embodiment, the fusion module uses the centroid analysis to detect output agreement based, for example, on the KNN algorithm although other approaches may be used.

In a further embodiment, the fusion module 315A determines when agreement is present between the analysis modules 310.

Each detected R peak provided by the analysis modules 310 will exceed a minimum threshold set based on the refractory period, which in at least one embodiment is defined as 240 milliseconds. For the R peak to be considered, the time difference between it and the previous quantitative decision from that detection algorithm module will need to exceed the minimum threshold. The R peaks are received by an input buffer in the fusion module 315A to allow for a set to be accumulated such that if each analysis module provides a respective R peak before any one of the detection algorithm modules provides two consecutive R peaks, then all of the R peaks in the input buffer are fused together and a determination is made for the current time frame.

However, if the input buffer receives two consecutive R peaks from one analysis module before receiving R peaks from all of the analysis modules for the current time period, then the received R peaks before the second of the consecutive R peaks are fused together and a determination is made for the current time frame.

During fusion, the fusion module determines whether all outputs (peak-to-peak intervals) are substantially equal, and if so, then a beat is detected and outputted by the fusion module. Otherwise, no beat is outputted by the fusion module.

In an alternative embodiment, the fusion module weighs other factors when not all individual outputs are substantially equal. Define a mode as the same output reported by at least two analysis modules. If there are two modes (i.e., two different pairs of analysis modules agreed), the first mode is outputted. If there is one mode, it is outputted. If there are no modes and multiple analysis modules reported a beat, the output interval closest to the average of the previous twelve final outputs is returned as the new final output. In alternative embodiments, the previous five, seven, ten, or fifteen final outputs are used instead of the previous twelve final outputs. Otherwise, no output is provided by the fusion module.

In at least one embodiment, the scoring module and the fusion module are examples of an aggregation module.

In at least one embodiment, the HRV/HRC module uses the provided signal from the scoring module (or fusion module depending on the particular embodiment) to provide an indication of HRV and/or HRC. In an alternative embodiment, the HRV/HRC module also provides a confidence level for the indication based on a bSQI value. See infra.

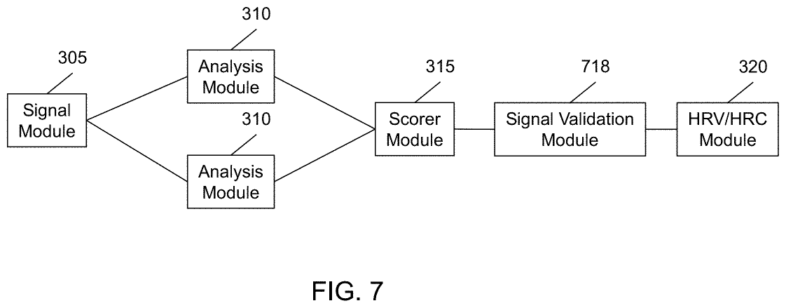

In an alternative embodiment, a signal validation module 718 is placed between the scoring module 315 and the HRV/HRC module 320 to validate the selection of the analysis module 310 as illustrated in FIG. 7. A comparison of how well individual algorithms performed within a given time frame provides one estimate of the level of noise in a signal. The beat detection signal quality (bSQI) of a waveform, with a time frame of N_{Total} beats, was defined to be the ratio of beats detected synchronously by n peak detection algorithms to all the beats detected by the final detection algorithm:

bSQI=(N_{matched}/N_{Total})

where N_{Matched} denotes the number of beats (or isolated events) agreed by a specified number n of algorithms, N_{Total} denotes both the time frame and beats detected by the final detection algorithm, and bSQI denotes the waveform's beat SQI. Following fusion at the detection level, outputs from the analysis module with the highest bSQI are selected for the HRV/HRC calculation. In at least one implementation, whenever a chosen number of analysis modules detecting the same peak of a waveform, a match was recorded. The higher the number of matches within a specified time window, the higher the beat signal quality.

In an alternative embodiment, the system further includes an alarm module connected to at least one medical sensor for triggering an alarm when the signal from that at least one medical sensor leaves a predetermined range.

### Experimental Information

During the course of development of the system, individual detection algorithms have been tested and compared to the results produced by a prototype system built according to at least one embodiment of the invention.

To provide a basis of comparison and improvement in R-wave detection (RWD) by at least one embodiment of the invention, a study was conducted to compare one embodiment to existing RWD algorithms based upon (1) their individual performance—as measured using two well-known benchmark parameters (sensitivity [Se], positive predictive value [+P]) against Physionet's Massachusetts Institute of Technology-Beth Israel Hospital (MIT-BIH) Arrhythmia Database and as published in the literature and (2) their ease of implementation for real-time computation. Ease of implementation denoted how well the mechanisms of a detection algorithm were understood. The selected algorithms were the Pan-Tompkins (Se: 99.57%, +P: 99.76%) [Pan, J. et al., A Real-Time QRS Detection Algorithm, IEEE Trans Biomed Eng, 32:230-236 (1985)], Hamilton-Tompkins (Se: 99.69%, +P: 99.77%) [Hamilton, P. S. et al., Quantitative Investigation of QRS Detection Rules Using the MIT/BIH Arrhythmia Database, IEEE Trans Biomed Eng, 33:1157-1165 (1986)], Christov (Se: 99.74%, +P: 99.65%) [Christov II, Real time electrocardiogram QRS detection using combined adaptive threshold, BioMedical Engineering OnLine, 3:1-9 (2004)], Afonso-Tompkins-Nguyen-Luo (Se: 99.59%, +P: 99.56%) [Afonso, V. X. et al, ECG Beat Detection Using Filter Banks, IEEE Trans Biomed Eng, 46:192-202 (1999)], and Zong-Moody-Jiang (Se: 99.65%, +P: 99.77%) [Zong, W. et al., A robust open-source algorithm to detect onset and duration of QRS complexes, Computers in Cardiology, 30:737-740 (2003)] algorithms.

Each of the algorithms (although the first two Tompkins algorithms were combined together) was adapted to be platform-independent and operable for real-time output. The algorithms were implemented in the Java (Sun Microsystems, Sunnyvale, Calif.) programming language using the Eclipse Integrated Development Environment (Eclipse Foundation, Ottawa, Canada).

Patient data and clinical validation used 250 ICU patients, as described by records in the Massachusetts General Hospital/Marquette Foundation (MGH/MF) Waveform Database, were selected for this study. Of these patients, 155 were males, 77 were females, and 18 were not specified. In addition, 20 patients had atrial fibrillation (AF), 65 had sinus tachycardia (ST), 111 had normal sinus rhythm (NSR), and the remaining patients had other rhythms, such as sinus bradycardia or ventricular pacing. Demographics of patients are shown in Table 2.

TABLE 2

| | Total | | Age | | HR | | High ABP | | Low ABP | |
|---|---|---|---|---|---|---|---|---|---|---|
| Patients | # | % | mean | std | mean | std | mean | std | mean | std |
| Entire database | 250 | 100.0 | 58.4 | 22.0 | 91.4 | 19.4 | 127.3 | 27.8 | 58.4 | 14.7 |
| Gender | | | | | | | | | | |
| Females | 77 | 30.8 | 57.1 | 21.4 | 91.6 | 18.6 | 126.6 | 28.2 | 58.8 | 13.6 |
| Males | 155 | 62.0 | 59.1 | 22.4 | 91.4 | 19.9 | 127.6 | 27.7 | 58.2 | 15.2 |
| Unknown | 18 | 7.2 | — | — | — | — | — | — | — | — |
| Underlying rhythm | | | | | | | | | | |
| Atrial | 20 | 8.0 | 73.3 | 9.2 | 91.2 | 13.6 | 118.6 | 24.8 | 53.8 | 9.8 |
| fibrillation | | | | | | | | | | |
| Atrial flutter | 4 | 1.6 | 72.0 | 6.1 | 64.3 | 12.2 | 147.5 | 25.0 | 46.0 | 4.9 |
| Atrial pacing | 5 | 2.0 | 66.4 | 9.7 | 89.6 | 8.9 | 154.8 | 38.9 | 56.2 | 24.8 |
| Atrial tachycardia | 1 | 0.4 | 0.8 | — | — | — | — | — | — | — |
| Atrioventricular | 1 | 0.4 | 73.0 | — | 89.0 | — | 134.0 | — | 31.0 | — |
| pacing | | | | | | | | | | |
| Chaotic atrial | 2 | 0.8 | 70.0 | 1.4 | 80.0 | | 126.0 | 8.5 | 56.0 | 8.5 |
| rhythm | | | | | | | | | | |
| Dual chamber | 1 | 0.4 | 52.0 | — | — | — | — | — | — | — |
| Junctional escape | 1 | 0.4 | 80.0 | — | 140.0 | — | 50.0 | — | 56.0 | — |
| rhythm | | | | | | | | | | |
| Junctional rhythm | 2 | 0.8 | 60.0 | — | 96.0 | — | 110.0 | — | 70.0 | — |
| Junctional | 1 | 0.4 | 55.0 | — | 103.0 | — | 168.0 | — | 10.0 | — |
| tachycardia | | | | | | | | | | |
| Multifocal atrial | 1 | 0.4 | 77.0 | — | 120.0 | — | 123.0 | — | 54.0 | — |
| tachycardia | | | | | | | | | | |
| Sinus arrhythmia | 1 | 0.4 | 76.0 | — | 66.0 | — | 160.0 | — | 80.0 | — |
| Sinus bradycardia | 6 | 2.4 | 67.3 | 1.2 | 50.8 | 8.2 | 172.0 | 27.8 | 44.0 | 32.9 |
| Sinus rhythm | 111 | 44.4 | 13.3 | 18.8 | 9.0 | 10.7 | 20.8 | 26.1 | 9.7 | 12.4 |
| Sinus tachycardia | 65 | 26.0 | 50.7 | 27.2 | 113.3 | 11.1 | 120.9 | 27.7 | 59.6 | 15.4 |
| Unknown | 20 | 8.0 | 0.2 | 0.3 | — | — | — | — | — | — |
| Ventricular pacing | 8 | 3.2 | 62.5 | 17.8 | 78.0 | 21.7 | 116.3 | 20.5 | 60.6 | 9.4 |

Selection of the MGH/MF Waveform Database was based upon the following considerations. First, this database was developed to extend the scope of the MIT-BIH Arrhythmia Database, which has been historically utilized much for beat detection and in previous work. The MGH/MF Waveform's Database's similarity to and improvements over its predecessor were desirable, such as the availability of simultaneous hemodynamic data and multiple ECG leads. Second, all records were easily accessible and documented, and none were excluded from the study; this would not have been made possible by larger and/or more recent sources. Third, because of the patients' broad demographics (see underlying rhythms in Table 2), it was possible to obtain a wide range of HRC values needed for analysis and comparison without filtering records. Lastly, online documentation simplified the task of classifying patients into groups. These considerations made it more suitable for the use of the MGH/MF Waveform Database over other sources.

Using simultaneously acquired ECG and ABP waveforms from these records, the data fusion framework was evaluated. Only three ECG leads (Leads I, II, V) were available, and all waveforms were sampled at 375 Hz. The dominant lead was Lead II. Validation involved over 375 hours of patient data describing patients with a broad spectrum of conditions. Individual recordings varied in length from 12 to 86 minutes, and in most cases, were about an hour long.

Evaluation of the data fusion framework began with calculation of mean entropy values for each set of individual SampEn values corresponding to records of the MGH/MF Waveform Database. Data analysis was performed by applying a sliding window of 200 PPIs (N=200, m=2, r=6) to interval sequences either manually verified or detected from one of the waveforms mentioned above (ECG Leads I, II, V and ABP). Thus, it was possible to obtain 5×250=1250 mean values, 250 values coming from manually verified sequences in the database. Furthermore, individual SampEn values were calculated across every record using the fusion of ECG Leads I, II, V, and ABP and then obtained mean entropy values. The gold standard for validation was manual verification of R waves, which was accomplished by manually picking times of R waves on time points of the ECG. After hand-picking R waves of all human records, times and RRIs were written to text files for future reference.

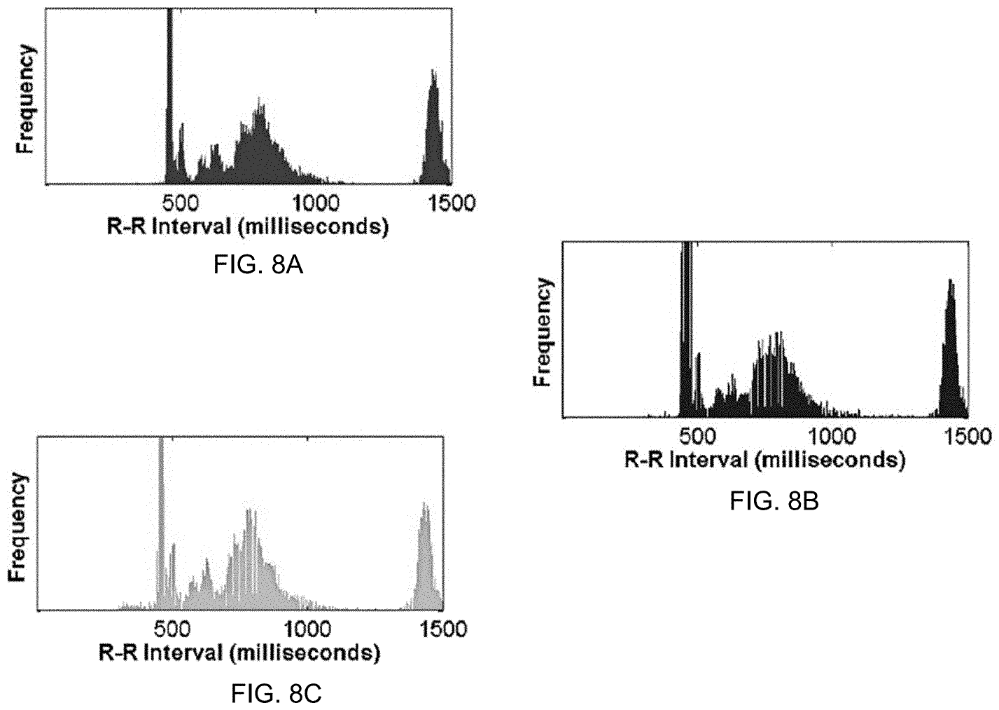

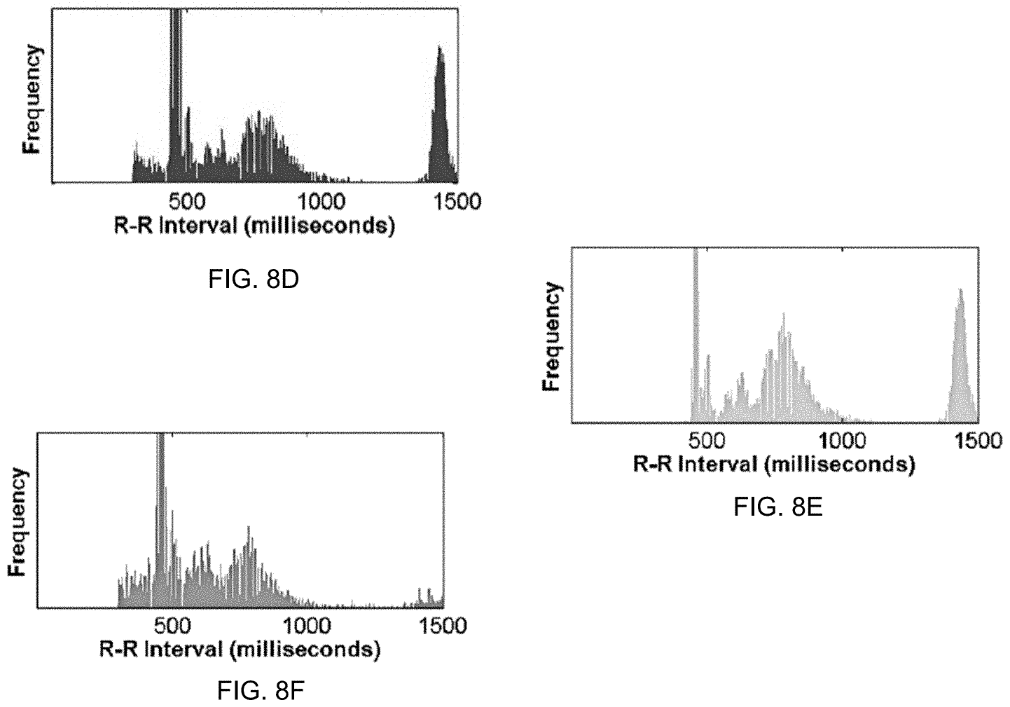

The parameters Se and +P were used for statistical analysis to compare the detection performances of our data fusion framework against individual waveforms and a combination of the four available waveforms (see Table 3). Further comparisons were made using histogram plots, which are depicted in FIGS. 8A-8F. In addition, mean HRC values were calculated for all records, and paired t-tests (in which the null hypothesis was that no difference exists between groups) as well as equivalence tests (two one-sided t-tests) were then performed in order to compare values derived from automatically detected peaks (“Fusion”, Lead I, Lead II, Lead V, and ABP) with those derived from manually verified sequences (see Tables 4 and 5, respectively). JMP version 9.0.0 (SAS Institute, Cary, N.C.) and the R Language (obtained from http://www.r-project.org/), which is a well-known open-source statistical software package, were used for statistical analysis.

TABLE 3

| Waveform | Verified | TP | FP | FN | Se (%) | +P (%) | Avg(Se, +P) |
|---|---|---|---|---|---|---|---|
| Fusion | 1526672 | 1382804 | 47236 | 143868 | 90.6 | 96.7 | 93.7 |
| Lead I | 1526672 | 1245965 | 297559 | 280707 | 81.6 | 80.7 | 81.2 |
| Lead II | 1526672 | 1433281 | 122752 | 93391 | 93.9 | 92.1 | 93.0 |
| Lead V | 1526672 | 1392478 | 161615 | 134194 | 91.2 | 89.6 | 90.4 |
| ABP | 1526672 | 1022140 | 443551 | 504532 | 67.0 | 69.7 | 68.4 |

In Table 3, ABP: Arterial Blood Pressure; TP: True Positive; FP: False Positive; FN: False Negative; Se: Sensitivity; +P: Positive Predictive Value, Avg(Se,+P): Average of Se and +P.

TABLE 4

| (p-value) | Mean | Fusion | Lead I | Lead II | Lead V | ABP |
|---|---|---|---|---|---|---|
| Manual | 0.84 ± 0.59 | 0.83 ± 0.59 | 0.88 ± 0.58 | 0.90 ± 0.58 | 0.86 ± 0.59 | 1.43 ± 0.54 |
| | | p = 0.06 | p < 0.001 | p < 0.001 | p = 0.02 | p < 0.0001 |

TABLE 5

| | Fusion | Lead I | Lead II | Lead V | ABP |
|---|---|---|---|---|---|
| Diff in means | 0.010 | −0.041 | −0.055 | −0.014 | −0.583 |
| Std err of | 0.053 | 0.053 | 0.053 | 0.054 | 0.051 |
| diff | | | | | |
| Max p-value | p = 0.045 | p = 0.133 | p = 0.201 | p = 0.056 | p = 1.000 |
| 1-α Cl for | 0.078 0.097 | −0.100 0.018 | −0.143 0.031 | −0.103 0.074 | −0.667 -0.498 |
| diff | | | | | |

In Table 5, Diff: difference; Std err: standard error; 1-α CI: 1-alpha confidence interval; α: test size=0.05; specified difference threshold=0.1; confidence level=0.9.

Finally, differences were analyzed by paired t-tests in order to determine whether statistical significances existed between errors from the “fusion” of waveform sources and each of the single waveform sources (see Table 6).

TABLE 6

| (p-value) | Error | Lead I | Lead II | Lead V | ABP |
|---|---|---|---|---|---|
| Fusion | −0.01 ± 0.35 | 0.04 ± 0.47 | 0.05 ± 0.33 | 0.2 ± 0.34 | 0.57 ± 0.68 |
| | | p = 0.01 | p = 0.02 | p = 0.4 | p < 0.0001 |

The Automated Electrocardiogram Selection of Peaks (AESOP) approach was developed in part to implement the fusion functions for merging ECG peak results from individual algorithms in real time. In at least one embodiment, this fusion approach employs four R-wave detectors as inputs and returns final detected peaks, corresponding times, and beat signal quality indices as outputs in approximate real time. Similar to a nearest-neighbor selection scheme, the AESOP approach in at least one embodiment selects the end time corresponding to a mode RRI or the RRI closest to the previous averaged 12 RRIs. In other words, if two or more component algorithms detect the same ECG peak, the AESOP approach selects the end time and peak value corresponding to the mode RRI's end time. Otherwise, the end time and ECG peak yielding an RRI closest to the previous averaged 12 RRIs is selected; this number (12) was chosen based upon heuristics in order to ensure a reasonable average. The AESOP approach required less than 6 seconds to analyze one record of the MIT-BIH Arrhythmia Database on an Intel® Core™ Duo central processing unit at 2.93 GHz.

Similarly, the Bypassing Electrocardiogram Beats or Peaks (BEBOP) approach was developed for merging non-ECG peak results. In at least one embodiment, the BEPOP approach differs from the AESOP approach in two of its sub-implementations, namely, that two algorithms in the AESOP approach were replaced with other algorithms, one version for detecting only positive-negative slope deflections in order to focus on systole dynamics and the other for detecting positive-negative slope deflections of a first-order derivative of the original signal.

For 250 ICU patient records in the MGH/MF Waveform Database, the data fusion framework (AESOP, BEBOP) achieved an averaged Se and +P of 93.7%, thereby outperforming results for individual waveforms in terms of mean Se/+P, i.e., tradeoff between Se and +P (see Table 3 supra). In terms of operating points, out of 1526672 true beats, the framework detected 1382804 TPs, 47236 FPs, and 143868 FNs (see Table 3 supra). Histogram plots of PPI sequences are shown in FIGS. 8A-8F. Importantly, paired t-tests (in which the null hypothesis was that no difference exists between mean HRC values derived from manually verified sequences and those derived from automatically detected peaks) showed that the “Fusion” values were the least statistically different from the gold standard (see Table 4 supra). Furthermore, using 0.1 as the difference considered practically zero, equivalence tests showed that only the “Fusion” values were practically equivalent to the gold standard (see Table 5 supra). Lastly, the fusion of waveform sources produced better error density distributions than those derived from individual waveforms as well as narrower confidence intervals. Statistical significances were determined for all error comparisons (p<0.05), except between “Fusion” and Lead V (p=0.35), with the most significance between the “fusion” of waveform sources and ABP (p<0.001).

Although this study involved a fairly standard dataset based upon availability of three ECG lead as well as arterial blood pressure waveforms, the dataset presented enough noisy waveforms to challenge the data fusion framework. Therefore, due to the quality of underlying data, the performance of R-wave detection reported in Table 3 was quite low. Nevertheless, better R-wave detection performance results in better signal-derived metrics (see Tables 3-5). It is important to note here that many previously published results of beat detection algorithms against different databases (e.g., MIT-BIH Arrhythmia Database) involved the detection of beats or QRS complexes, rather than detection of R-waves. Hence, their results may not reflect stringent requirements on performance and may be better suited for beat applications, as opposed to the real-time calculation of HRC.

An initial analysis was conducted to observe whether a fusion of three inferior waveforms produced results commensurate with those obtained from one reliable waveform (e.g., ECG Lead II); afterwards, data analysis involved all four waveforms (Leads I, II, V, and ABP). Importantly, the fusion of all available waveforms produced results better than those obtained from one reliable waveform (e.g., ECG Lead II). Visual comparisons of the error density distributions (histograms) superimposed on the left-hand sides of each Bland-Altman plot were made in order to determine whether the data fusion framework yielded the best results. The fusion of waveform sources produced slightly better error density distributions than those belonging to individual waveform sources (see Table 6). Therefore, the method for calculating HRC has much potential application in the clinical environment.

Statistical significances were found for all error comparisons (p<0.05), except between “Fusion” and Lead V (p=0.35), with the most significance between ABP and manual verification (p<0.001), suggesting that the ECG and ABP waveforms of patient records in the MGH/MF Waveform Database are disparate in quality. The fact that errors for Lead V were not significantly different from those for the fusion of waveform channels also demonstrated that the dominant lead (in this case, Lead II) may not always be reliable for signal-derived metrics. As expected, because of the ABP waveform's poor quality, the data fusion framework rejected use of this signal during fusion; this proved valuable for testing the framework. Had all waveforms been degraded in quality, a data fusion framework could prove optimal for calculating HRC in the ICU environment.

These observations agree with prior findings. Equivalence tests demonstrated the data fusion framework's overall reliability. While errors for Lead V were not significantly different from those for the fusion of waveform channels, this was not as apparent when considering equivalence between mean values. Because mean HRC values varied by rhythm, i.e., AF (1.0±0.9); NSR (0.9±0.5); and ST (0.7±0.5), from Tables 4 and 5, this shows that overall improved HRC calculations can better discriminate patient groups.

In a forthcoming publication in the Journal of Trauma and Acute Care Surgery entitled Is heart-rate complexity a surrogate measure of cardiac output before, during, and after hemorrhage in a conscious sheep module of multiple hemorrhages and resuscitation? regarding a study, it is observed that heart rate complexity (HRC) is a cardiac output (CO) surrogate before and during hemorrhage, but not during resuscitation, in a conscious sheep model of multiple hemorrhages and resuscitation. The results and observations from this study suggest that HRC may be used to detect rapid patient destabilization when standard vital signs cannot do so because of inherent physiologic compensatory mechanisms and further in view of the problems discussed previously in this disclosure. HRC is best calculated as accurately as possible and thus benefits from having high quality heartbeat information in which to analyze data from a patient.

As will be appreciated by one skilled in the art based on this disclosure, aspects of the present invention may be embodied as a system, method or computer program product. Accordingly, aspects of the present invention may take the form of an entirely hardware embodiment, a processor operating with software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this disclosure, a computer readable storage medium may be any tangible medium that can contain, or store a program for use by or in connection with an instruction execution system, apparatus, or device.

Computer program code for carrying out operations for aspects of the present invention may be written in any combination of one or more programming languages, including an object oriented programming language such as Java, Smalltalk, C++, C#, Transact-SQL, XML, or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute with the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

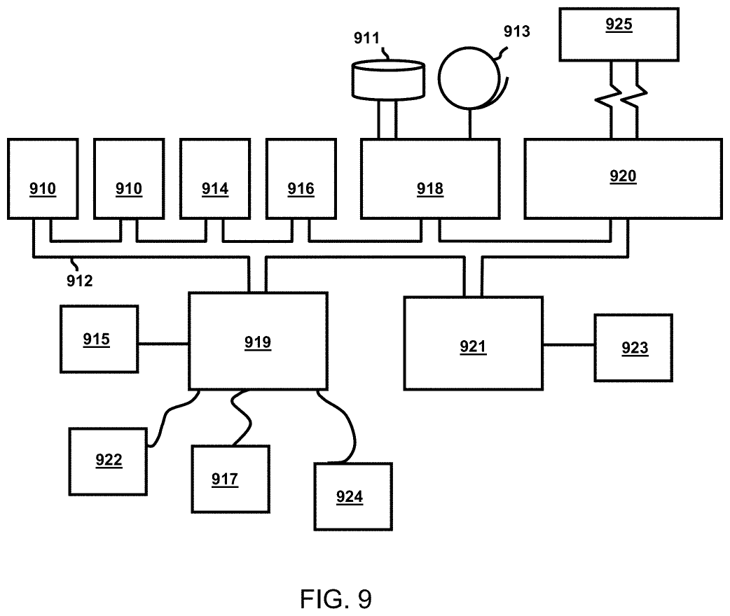

Referring now to FIG. 9, a representative hardware environment for practicing at least one embodiment of the invention is depicted. This schematic drawing illustrates a hardware configuration of an information handling/computer system in accordance with at least one embodiment of the invention. The system comprises at least one processor or central processing unit (CPU) 910. The CPUs 910 are interconnected with system bus 913 to various devices such as a random access memory (RAM) 915, read-only memory (ROM) 916, and an input/output (I/O) adapter 918. The I/O adapter 918 can connect to peripheral devices, such as disk units 911 and tape drives 914, or other program storage devices that are readable by the system. The system can read the inventive instructions on the program storage devices and follow these instructions to execute the methodology of at least one embodiment of the invention. The system further includes a user interface adapter 919 that connects a keyboard 915, mouse 917, speaker 935, microphone 933, and/or other user interface devices such as a touch screen device (not shown) to the bus 913 to gather user input. Additionally, a communication adapter 930 connects the bus 913 to a data processing network 935, and a display adapter 931 connects the bus 913 to a display device 834 which may be embodied as an output device such as a monitor, printer, or transmitter, for example.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

Although the present invention has been described in terms of particular example embodiments, it is not limited to those embodiments. The embodiments, examples, and modifications which would still be encompassed by the invention may be made by those skilled in the art, particularly in light of the foregoing teachings.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

### APPENDIX

Sample, Quadratic Sample, Multiscale Entropy

The sample entropy, SampEn(m, r), equals the negative natural logarithm of the conditional probability that two epochs similar for m RRIs remain similar at the next RRI, given a sequence of N RRIs and excluding self-matches. Here, similarity means that RRIs differ by no more than some tolerance r, be it seconds or milliseconds.

For clarity, sample entropy may be computed by the following equations:

SampEn(m,r,N)=−ln(A/B), (1)

B=[(N-m-1)/2]Σᵢ₌₁^{N-m}Bᵢ^{r}(m), (2)

A=[(N-m-1)/2]Σᵢ₌₁^{N-m}Aᵢ^{r}(m). (3)

In other words, for a sequence of N intervals, if xₘ(i) is an epoch of m consecutive intervals starting at index i and running from i=1, . . . , N-m, then Bᵢ^{r} (m) denotes the number of epochs xₘ(j) within r of xₘ(i), for i≠j, multiplied by (N-m-1)⁻¹, and A^{r}ᵢ(m) denotes the number of epochs xₘ₊₁(j) within r of xₘ₊₁(i), for i≠j, multiplied by (N-m-1)⁻¹.

The quadratic sample entropy, QSE(m, r), equals SampEn(m, r) normalized by 2r. Obtaining a coarse-grained RRI sequence from the original sequence and calculating SampEn(m, r) produces the multiscale entropy, MSE(m, r, s), where s denotes a scaling factor used in the coarse-graining equation

$${y}_{j} = \frac{1}{s} {\sum }_{i = ( j - 1 ) s + 1}^{js} {x}_{i} , \qquad ( 4 )$$

∀ integers j such that 1≤j≤N/s, xᵢ denotes the i^{th} RRI of the original sequence, and yⱼ denotes the j^{th} RRI of the coarse-grained sequence.

Poincaré Variability Ratio

The Poincaré plot takes an RRI sequence and plots each RRI against the following RRI. A quantitative analysis of the plot's geometry involves the calculation of two standard deviations, SD1 and SD2, respectively, describing the short-term and long-term RRI variability of a sequence, as well as calculation of the variability ratio SD1/SD2. Because SD1 and SD2 correspond to the minor and major axes, respectively, of an ellipse fitted over the scatter plot, they can be determined numerically by calculating the standard deviations of distances from each point on the plot to the lines RRIᵢ₊₁=RRIᵢ and RRIᵢ₊₁=−RRIᵢ+2RRIₘₑₐₙ, respectively. Alternatively, they can be obtained from the following equations:

$$\begin{matrix}SD {1}^{2} = var ( \frac{1}{\sqrt{2}} {RRI}_{i} - \frac{1}{\sqrt{2}} {RRI}_{i + 1} ) = \frac{1}{2} {SDSD}^{2} , \\ and & ( 5 ) \\ SD {2}^{2} = 2 {SDRR}^{2} - \frac{1}{2} {SDSD}^{2} , & ( 6 )\end{matrix}$$

∀i=1, . . . , N, where var(•) denotes the variance function, SDRR denotes the standard deviation of RRIs, SDSD denotes the standard deviation of successive RRI differences, and N denotes the number of RRIs in the sequence window.

Fractal Scaling Exponent

Detrended fluctuation analysis (DFA) quantifies the long-range correlation of RRI sequences by estimating the fractal scaling exponent α within a sequence. The following steps are used to perform DFA on an RRI sequence with N RRIs. Integrate the RRI sequence to obtain y(k), where k denotes the index of an RRI in the original sequence. Divide y(k) into boxes of length n, for some box size n. For each box, calculate a least squares line yₙ(k) best fitted to y(k), the line itself representing the trend in that box. Calculate the root-mean-square (RMS) fluctuation of the integrated and detrended sequence using the equation

$$F ( n ) = \sqrt{\frac{1}{N} {\sum }_{k = 1}^{N} {[ y ( k ) - {y}_{n} ( k ) ]}^{2}} . \qquad ( 7 )$$

Repeat the second step through the fourth step over all time scales (box sizes) in order to characterize the relationship between log F(n) and log n. Obtain the slope a of the line relating log F(n) and log n.

Here, the linear relationship between F(n) and n on a log-log scale describes the degree of power law (fractal) scaling within y(k). In other words, the scaling exponent a indicates the roughness of the original RRI sequence. The larger the value of α, the smoother the RRI sequence. According to this method, α≈0.5 implies that a sequence resembles white Gaussian noise (a totally random sequence), while α≈1.0 implies that the sequence resembles a fractal-like time series. Analogously, α≈1.5 implies that a sequence resembles Brownian noise with decreasing power in its high frequency components.

In addition, suitable values of n for estimating the scaling exponent α include 4≤n≤N/4 uniformly distributed over log n. Another form of analysis is to estimate a short-term scaling exponent α₁ for 4≤n≤16 and a long-term scaling exponent α₂ for 16≤n≤64. Although these techniques make DFA more applicable to both large and relatively short data sets, in general, larger data lengths and the editing of ectopic beats will improve data analysis.

Autocorrelation Coefficient

The autocorrelation coefficient utilizes the histogram. Given a defined RRI window, this coefficient calculates the overlap of the histograms for two consecutive RRI windows according to the equation

$$A ( k , τ ) = {\sum }_{bin = 1}^{{N}_{bins}} [ {p}_{bin} ( k ) {p}_{bin} ( k + τ ) ] , \qquad ( 8 )$$

where k denotes the starting index of an RRI in the original windowed sequence, r denotes a shift from the starting index, p_{bin}(k) denotes the probability that an RRI falls into the designated bin of the histogram probability distribution, and N_{bins} denotes the number of bins in the histogram. Since the coefficient A(k, τ), sometimes referred to as the similarity of distributions (SOD), measures a probability, it falls into the range of [0, 1]. In particular, it tends toward 0 if the RRI histograms have non-zero values dispersed across all bins, and it tends towards 1 if the histograms have similar peaks. Hence, the SOD measures the predictability and stability of the heart's dynamics at a specific time.

Degree of Nonstationarity

The index StatAv measures the degree of nonstationarity within an RRI sequence. Formally, StatAv breaks up a given RRI window into 40 epochs, computes the sample mean of each epoch, and then calculates the ratio between the standard deviation of the 40 means and the SDRR. Given N RRIs in an RRI window, this can be expressed mathematically as

StatAv=—SDAV/SDRR (9)

where SDAV denotes the standard deviation of M₁, M₂, . . . , M₄₀ and Mₖ denotes the sample mean of an epoch formed inclusively from RRI; between i=N•(k-1)/40+1 and N•k/40, ∀k=1, . . . 40. Based upon this definition, smaller StatAv values imply more stationarity. Thus, StatAv quantifies the tendency of the mean to vary with time.

What is claimed is:

1. A method for providing a beat determination based on signals from a plurality of medical sensors attached to one patient in substantially real time, said method comprising:
receiving in at least two analysis modules at least one signal from at least one medical sensor, wherein the signal includes at least one of an ECG signal and a pulse signal such that at least one analysis module receives the one ECG signal and at least one analysis module receives the one pulse signal;
in each analysis module
processing the one signal through a plurality of detection algorithm modules with each detection algorithm module producing at least one quantitative decision,
fusing the quantitative decisions from the detection algorithm modules together with a fusion module, and
outputting at least one detected peak from the fusion module to an aggregation module in communication with the at least two analysis modules; and
producing from the aggregation module a series of detected peaks representing heartbeats of the patient based on a series of detected peaks received from the analysis modules.

2. The method according to claim 1, wherein producing the series of detected peaks is further based on a beat signal quality index for each analysis module with the analysis module with the highest beat signal quality index being selected to supply the detected peaks for output by the aggregation module.

3. The method according to claim 2, wherein the beat signal quality index equals the beats detected by that analysis module over a predetermined time period divided by an average of the total number of beats detected by all of the analysis modules over the predetermined time period.

4. The method according to claim 1, wherein fusing the quantitative decisions together includes
receiving quantitative decisions in a buffer of the fusion module from the decision algorithm modules in communication with the fusion module;
determining whether quantitative decisions have been received from all of the decision algorithm modules;
determining whether a most recently received quantitative decision is a second consecutive decision from one detection algorithm module;
when either determination is true,
fusing the received quantitative decisions, and
determining an existence of a peak and outputting any peak that exists; and
when neither determination is true, waiting for the next quantitative decision to be received from the detection algorithm modules.

5. The method according to claim 4, wherein fusing the quantitative decisions occurs after elapse of a refractory period after the last peak is determined to exist.

6. The method according to claim 1, wherein each detection algorithm module
pre-processes the one signal to at least one of filter the signal and extract features from the signal to provide at least one processed signal, and
analyzes the at least one processed signal to detect whether a beat is present in the processed signal.

7. The method according to claim 6, wherein at least one analysis module includes at least two detection algorithm modules that share at least one processed signal.

8. The method according to claim 1, wherein aggregating by the aggregation module includes
receiving detected peaks in a buffer from the analysis modules;
determining whether detected peaks have been received from all of the analysis modules;
determining whether a most recent received detected peak is a second consecutive detected peak from one analysis module;
when either determination is true,
fusing the received detected peaks, and
determining an existence of a peak and outputting any peak that exists; and
when neither determination is true, waiting for the next detected peak to be received from the analysis modules.

9. The method according to claim 8, wherein fusing the received detected peaks by the fusion module includes analyzing the detected peaks using centroid analysis to detect output agreement.

10. The method according to claim 9, wherein output agreement is based on K-nearest neighbor.

11. The method according to claim 1, further comprising:
receiving the series of detected peaks from the aggregation module into a heart rate complexity module;
determining heart rate complexity by the heart rate complexity module based on the received detected peaks; and
triggering an alarm when the heart rate complexity is outside a predetermined range.

12. The method according to claim 11, wherein the heart rate complexity based on an entropy measure.

13. The method according to claim 1, further comprising selecting the analysis module with a highest best signal quality index using a signal validation model.

14. The method according to claim 1, wherein the receiving analysis module is selected based on the detection algorithm modules present in the receiving analysis module being configured to process the at least one signal.

15. The method according to claim 1, wherein each detection algorithm module is configured to use different logic to detect a beat in the input signal as the quantitative decision.

16. The method according to claim 1, wherein the plurality of detection algorithm modules in each analysis module numbers at least four.

17. The method according to claim 1, wherein the plurality of medical sensors includes at least one ECG lead and a pulse oximeter.

18. A method for providing a beat determination based on signals from a plurality of medical sensors attached to one patient in substantially real time, said method comprising:
receiving in at least two analysis modules at least one signal from at least one medical sensor, wherein the signal includes at least one of an ECG signal and a pulse signal such that at least one analysis module receives the one ECG signal and at least one analysis module receives the one pulse signal;
in each analysis module
processing the one signal through a plurality of detection algorithm modules with each detection algorithm module producing at least one quantitative decision,
fusing the quantitative decisions from the detection algorithm modules together with a fusion module, and
outputting at least one detected peak from the fusion module to an aggregation module in communication with the at least two analysis modules; and
producing from the aggregation module a series of detected peaks representing heartbeats of the patient based on a series of detected peaks received from the analysis module having a highest beat signal quality index for each analysis module with the analysis module with the highest best signal quality index being selected to supply the detected peaks for output by the aggregation module, and
wherein the beat signal quality index equals the beats detected by that analysis module over a predetermined time period divided by an average of the total number of beats detected by all of the analysis modules over the predetermined time period.

19. The method according to claim 18, wherein
fusing the quantitative decisions together includes
receiving quantitative decisions in a buffer of the fusion module from the decision algorithm modules in communication with the fusion module;
determining whether quantitative decisions have been received from all of the decision algorithm modules;
determining whether a most recent received quantitative decision is a second consecutive decision from one detection algorithm module;
when either determination is true,
fusing the received quantitative decisions, and
determining an existence of a peak and outputting any peak that exists; and
when neither determination is true, waiting for the next quantitative decision to be received from the detection algorithm modules,
fusing the quantitative decisions occurs after elapse of a refractory period after the last peak is determined to exist,
each detection algorithm module
pre-processes the one signal to at least one of filter the signal and extract features from the signal to provide at least one processed signal,
analyzes the at least one processed signal to detect whether a beat is present in the processed signal, and
at least two detection algorithm modules in at least one of the at least two analysis modules share at least one processed signal.

20. A computer program product for detecting peaks in a plurality of biomedical signals, the computer program product comprising:
a computer readable storage medium having encoded thereon:
first program instructions executable by a processor to cause the processer to receive a plurality of biomedical signals originating from a plurality of medical sensors;
second program instructions executable by a processor to cause the processer to assign each signal to a particular analysis module based on a type of signal that the signal is;
third program instructions executable by a processor to cause the processer to use each analysis module to analyze one signal with a plurality of algorithms;
fourth program instructions executable by a processor to cause the processer to use each analysis module to fuse the outputs of the plurality of algorithms to provide an output;
fifth program instructions executable by a processor to cause the processer to select the highest quality output from the plurality of analysis modules; and
sixth program instructions executable by a processor to cause the processer to use the selected output to determine at least one of heart rate complexity and heart rate variability.
